(19) United States
(12) Patent Application Publication
(10) Pub. No.: US 2022/0122708 A1
(43) Pub. Date: Apr. 21, 2022

(54) IDENTIFICATION ASSISTANCE SYSTEM, IDENTIFICATION ASSISTANCE CLIENT, IDENTIFICATION ASSISTANCE SERVER, AND IDENTIFICATION ASSISTANCE METHOD

(71) Applicant: FUJIFILM Toyama Chemical Co., Ltd., Tokyo (JP)

(72) Inventors: Koki NAGATANI, Tokyo (JP); Takao UNE, Tokyo (JP)

(73) Assignee: FUJIFILM Toyama Chemical Co., Ltd., Tokyo (JP)

(21) Appl. No.: 17/564,415

(22) Filed: Dec. 29, 2021

Related U.S. Application Data
(63) Continuation of application No. PCT/JP2020/025508, filed on Jun. 29, 2020.

(30) Foreign Application Priority Data
Jul. 5, 2019 (JP) 2019-125891

(51) Int. Cl. G16H 20/10; G06N 7/02; G06V 30/19; G16H 70/00; G10L 15/22

(52) U.S. Cl. CPC G16H 20/10; G06N 7/023; G06V 30/19093; G10L 15/22; G06V 30/19193; G16H 70/00; G06V 30/191

(57) ABSTRACT

The present invention aims to provide an identification assistance system, an identification assistance client, and an identification assistance method that enable the user to identify drugs accurately and easily. In the identification assistance system according to an aspect of the present invention, first text which is the result of voice recognition is corrected, and thus errors of the voice recognition can be corrected. In addition, the first text is corrected with reference to a drug search dictionary having learned expressions used for drug identification, and thus expressions unique to drug identification can be taken into consideration. The user can perform a search not only by using the code and/or the name of the drug but also by speaking aloud the external appearance information on the drug. Thus, even if the code and the name are unknown, the user can perform a search by using the external appearance information.

### CROSS-REFERENCE TO RELATED APPLICATIONS

The present application is a Continuation of PCT International Application No. PCT/JP2020/025508 filed on Jun. 29, 2020 claiming priority under 35 U.S.C § 119(a) to Japanese Patent Application No. 2019-125891 filed on Jul. 5, 2019. Each of the above applications is hereby expressly incorporated by reference, in its entirety, into the present application.

### BACKGROUND OF THE INVENTION

### 1. Field of the Invention

The present invention relates to an identification assistance system, an identification assistance client, an identification assistance server, and an identification assistance method regarding drugs.

### 2. Description of the Related Art

In medical sites such as hospitals and pharmacies, when drugs are provided to patients or brought in by patients, the drugs are audited. However, manual audits and identification take a long working time, and are heavy burdens on users (such as doctors and pharmacists). To address this, use of voice recognition for the audits and identification is conceivable. For example, Japanese Patent Application Laid-Open No. 2015-064672 (hereinafter, referred to as Patent Literature 1) describes that a user specifies names of drugs used in the medical site with his/her voice, and the specified drugs are registered in a list of drugs to be used. In addition, Japanese Patent Application Laid-Open No. 2016-218998 (hereinafter, referred to as Patent Literature 2) describes that names of drugs are recognized by voice recognition, and information on the recognized drugs is presented.

### CITATION LIST

Patent Literature 1: Japanese Patent Application Laid-Open No. 2015-064672

Patent Literature 2: Japanese Patent Application Laid-Open No. 2016-218998

### SUMMARY OF THE INVENTION

Conventional techniques such as Patent Literatures 1 and 2 do not consider errors in voice input and expressions unique to drug identification. They only simply “use voice input and voice recognition”. Thus, they do not reduce burdens on the users. The present invention has been made in light of such situations, and an object of the present invention is to provide an identification assistance system, an identification assistance client, and an identification assistance method that enable a user to identify drugs accurately and easily. Another object of the present invention is to provide an identification assistance server that can be used for drug identification.

To achieve the objects, an identification assistance system according to a first aspect of the present invention includes: a voice recognizing unit configured to recognize received voice and output a recognition result as first text; a text correcting unit configured to refer to a drug search dictionary having learned expressions used for drug identification, and correct the first text to generate second text; a drug database configured to store identification information including codes and/or names of drugs and external appearance information on the drugs, as text information in a state where the external appearance information is associated with the identification information; a searching unit configured to search the drug database using the second text as a keyword and obtain the identification information on at least one candidate drug that is a candidate of a drug indicated by the second text; and an outputting unit configured to output the identification information on the candidate drug.

In the first aspect, since the first text which is the result of the voice recognition is corrected, it is possible to correct errors of the voice recognition. In addition, since the first text is corrected with reference to the drug search dictionary having learned expressions used for drug identification, it is possible to consider expressions unique to drug identification. The user can perform a search not only by using the code and/or the name of the drug, but also by speaking aloud the external appearance information of the drug. Thus, even in a case where the code and the name are unknown, the user can perform a search using the external appearance information. In the first aspect, “the external appearance information” means information indicating characteristics of drugs that the user can visually recognize. Note that the number of keywords may be one, or may be two or more.

Thus, the first aspect enables the user to identify drugs accurately and easily. Note that the components of the system in the first aspect may be housed in one housing or may be divided and housed in a plurality of housings. Alternatively, a plurality of devices may be connected via a network so as to fulfill the components of the first aspect as a whole.

According to a second aspect, in the identification assistance system according to the first aspect, the drug search dictionary is a conversion dictionary in which words used for drug identification are registered as conversion candidates. Examples of “words used for drug identification” include numerical characters (numerals), alphabet letters, and pharmaceutical companies' names, trade names, or abbreviated names of pharmaceutical companies. Such information is attached to drugs in some cases by using imprints and/or printed letters, print on the package, attachment of labels, or by other methods. Therefore, when such information is registered into the conversion dictionary, it is possible to input intended words as search keywords.

According to a third aspect, in the identification assistance system according to the first or second aspect, the voice recognizing unit generates the first text, using a trained model built by machine learning performed using the identification information and the external appearance information as teacher data. Here, the trained model may be a trained model using a neural network.

According to a fourth aspect, in the identification assistance system according to any one of the first to third aspects, the searching unit performs a partial match search using the second text as the keyword and performs a fuzzy search depending on a result of the partial match search. Since a partial match search is performed in the fourth aspect, it is possible to perform a search even in a case where only a part of the code, the name and the external appearance information is known due to, for example, division of a tablet or a package or other reasons. Note that in the fourth aspect, for example, in a case where the number of search hits (the number of retrieved items) is smaller than or equal to a threshold or in a case where the number of searched hits is zero, it is possible to perform a fuzzy search.

According to a fifth aspect, in the identification assistance system according to the fourth aspect, the searching unit normalizes the second text to generate normalized text and uses the normalized text to perform the partial match search. As “normalization”, for example, the searching unit can perform conversion from uppercase letters to lowercase letters, from full-width characters to half-width characters, and from kanji characters (Chinese characters) and/or hiragana characters (rounded Japanese phonetic syllabary) to katakana characters (angular Japanese phonetic syllabary). In addition, a fuzzy search is effective when it is difficult to perform an effective search using a partial search because the voice recognition result is different from the intended character string.

According to a sixth aspect, in the identification assistance system according to the fourth or fifth aspect, in the fuzzy search, the searching unit calculates a similarity degree between the second text and third text that is text included in the text information, and regards a drug that corresponds to the third text whose similarity degree is larger than or equal to a threshold, as the candidate drug. In the sixth aspect, the searching unit may calculate the similarity degree by using the distance between pieces of text.

According to a seventh aspect, in the identification assistance system according to the sixth aspect, the searching unit extracts a character string having the same length as the second text out of the third text and calculates the similarity degree. Keywords from voice input are often shortened. Relatively, the shorter the text information is, the higher the similarity degree is calculated. Therefore, in a case where keywords are shortened, sometimes, an appropriate search result cannot be obtained. However, even in such a case, according to the seventh aspect, because a character string having the same length as the second text is extracted from the third text and the similarity degree is calculated, it becomes more likely to obtain an appropriate search result.

According to an eighth aspect, in the identification assistance system according to any one of the first to seventh aspects, the text correcting unit receives correction to the second text and causes additional learning of the drug search dictionary based on the received correction. In the eighth aspect, the additional learning improves search accuracy.

According to a ninth aspect, in the identification assistance system according to any one of the first to eighth aspects, the external appearance information includes at least one kind of information out of imprint information and/or printed-letter information, shape information, and color information on the drugs. The ninth aspect prescribes the specific details of the external appearance information. The shape information is information indicating, for example, whether the shape of a drug is a round shape, an oval shape, a tablet, a capsule, or other shapes, and the color information is information indicating, for example, whether a drug is white, blue, red, or of other colors.

According to a tenth aspect, in the identification assistance system according to any one of the first to ninth aspects, the outputting unit outputs a file including the identification information on a drug selected out of the candidate drugs.

According to an eleventh aspect, in the identification assistance system according to any one of the first to tenth aspects, the drug database stores identification information on the drugs and images of the drugs, in a state where the images are associated with the identification information, and the outputting unit outputs an image of the candidate drug with the image of the candidate drug associated with the identification information, to a display device. The eleventh aspect makes it easy for the user to visually determine whether the search and the identification are appropriate. Note that the image of the drug may be an image of the package (such as the PTP sheet) of the drug, instead of the drug itself.

To achieve the objects, an identification assistance client according to a twelfth aspect of the present invention includes: a voice recognizing unit configured to recognize received voice and output a recognition result as first text; a text correcting unit configured to refer to a drug search dictionary having learned expressions used for drug identification and correct the first text to generate second text; a client-side transmitting unit configured to transmit information indicating the second text to an identification assistance server; a client-side receiving unit configured to receive identification information on at least one candidate drug that is a candidate of a drug corresponding to the second text from the identification assistance server, the identification information including a code and/or a name of the drug; and an outputting unit configured to output the identification information. The twelfth aspect enables the user to identify drugs accurately and easily. Note that the identification assistance client according to the twelfth aspect may include the configurations according to the second to eleventh aspects.

To achieve the objects, an identification assistance server according to a thirteenth aspect of the present invention includes: a drug database configured to store identification information including codes and/or names of drugs and external appearance information on the drugs, as text information in a state where the external appearance information is associated with the identification information; a server-side receiving unit configured to receive text information on a drug from an identification assistance client; a searching unit configured to search the drug database using the text information as a keyword and obtain the identification information on at least one candidate drug that is a candidate of the drug indicated by the text information; and a server-side transmitting unit configured to transmit the obtained identification information to the identification assistance client. The identification assistance server according to the thirteenth aspect can be used for assisting drug identification by voice input. Note that the identification assistance server according to the thirteenth aspect may include the configurations according to the second to eleventh aspects. In addition, the identification assistance client according to the twelfth aspect and the identification assistance server according to the thirteenth aspect may be used to achieve a system the same as or similar to the identification assistance system according to the first aspect.

To achieve the objects, an identification assistance method according to a fourteenth aspect of the present invention includes: a voice recognizing step of recognizing received voice and outputting a recognition result as first text; a text correcting step of referring to a drug search dictionary having learned expressions used for drug identification and correcting the first text to generate second text; a searching step of searching, using the second text as a keyword, a drug database storing identification information including codes and/or names of drugs and external appearance information on the drugs, as text information, in a state where the external appearance information is associated with the identification information, and obtaining the identification information on at least one candidate drug that is a candidate of a drug indicated by the second text; and an outputting step of outputting the identification information on the candidate drug. According to the fourteenth aspect, as in the first aspect, it becomes possible for the user to identify drugs accurately and easily by voice input. Note that the identification assistance method according to the fourteenth aspect may include configurations (steps) corresponding to or similar to those in the second to eleventh aspects. In addition, a program for causing an identification assistance system or a computer to execute the identification assistance methods of these aspects, and a non-transitory recording medium in which computer-readable code of the program is recorded are also included in the aspects of the present invention.

Note that the identification assistance systems, the identification assistance client, the identification assistance server, and the identification assistance method of the foregoing aspects can be used for drug identification assistance and/or audit assistance.

As has been described above, the identification assistance system, the identification assistance client, and the identification assistance method according to the present invention enable the user to identify drugs accurately and easily. In addition, the identification assistance server of the present invention can be used for drug identification.

### BRIEF DESCRIPTION OF THE DRAWINGS

FIG. 1 is a diagram illustrating a configuration of an identification assistance system according to a first embodiment.

FIG. 2 is a functional block diagram of a processing unit.

FIG. 3 is a diagram illustrating information stored in a storing unit.

FIG. 4 is a flowchart illustrating processing of an identification assistance method according to the first embodiment.

FIG. 5 is a diagram illustrating a configuration of an identification assistance system according to a second embodiment.

FIG. 6 is a functional block diagram of a client processing unit.

FIG. 7 is a diagram illustrating information stored in a client storing unit.

FIG. 8 is a functional block diagram of a server processing unit.

FIG. 9 is a diagram illustrating information stored in a server storing unit.

FIG. 10 is a flowchart illustrating processing of an identification assistance method according to the second embodiment.

FIG. 11 is a flowchart illustrating the processing of the identification assistance method according to the second embodiment.

FIG. 12 is a flowchart illustrating the processing of the identification assistance method according to the second embodiment.

### DESCRIPTION OF THE PREFERRED EMBODIMENTS

Hereinafter, embodiments of an identification assistance system, an identification assistance client, an identification assistance server, and an identification assistance method according to the present invention are described in detail with reference to the attached drawings.

### First Embodiment

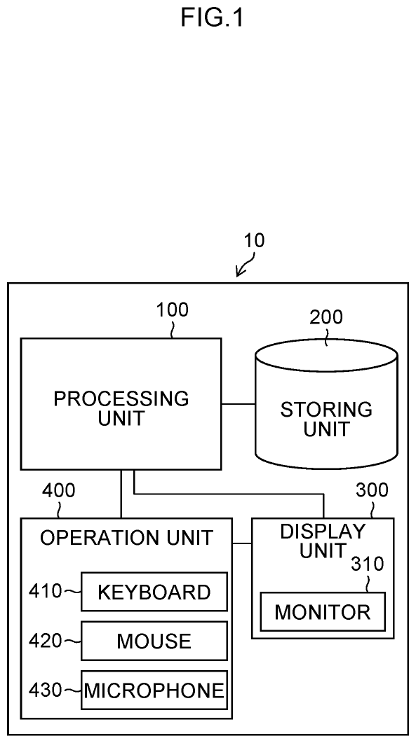

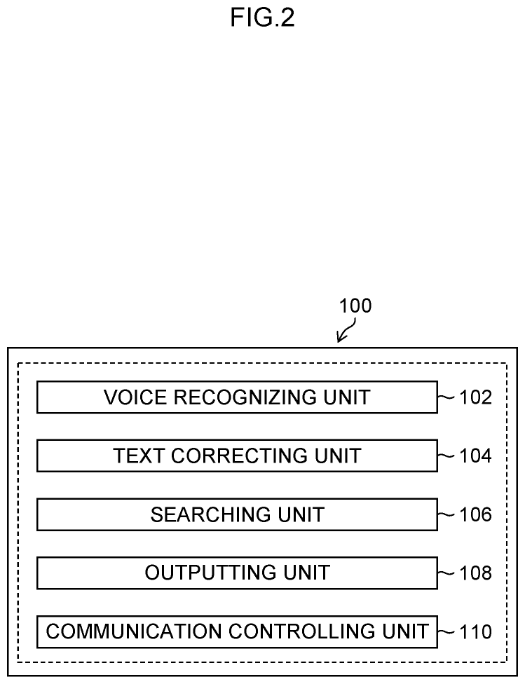

FIG. 1 is a block diagram illustrating the configuration of an identification assistance system 10 (identification assistance system) according to a first embodiment. The identification assistance system 10 is a system that assists drug identification and can be built by using a computer. As illustrated in FIG. 1, the identification assistance system 10 includes a processing unit 100, a storing unit 200, a display unit 300, and an operation unit 400. The components of the identification assistance system 10 are connected to one another so as to communicate necessary information between them. In addition, the identification assistance system 10 is connected to a not-illustrated external server, a not-illustrated external database and the like, via a communication controlling unit (communication controller) 110 (see FIG. 2) and a not-illustrated network, so as to obtain information as necessary.

Note that the identification assistance system 10 can be used for assisting identification of drugs or the like that are brought in by patients and audit of drugs that are to be provided to patients.

<Configuration of Processing Unit>

FIG. 2 is a diagram illustrating a configuration of the processing unit 100. The processing unit 100 includes a voice recognizing unit 102 (voice recognizing unit), a text correcting unit 104 (text correcting unit), a searching unit 106 (searching unit), an outputting unit 108 (outputting unit), and the communication controlling unit 110. The processing unit 100 further includes a not-illustrated central processing unit (CPU), read only memory (ROM), and random access memory (RAM). Note that processing by these units is performed under control of the CPU.

The function of each unit of the foregoing processing unit 100 can be implemented by using various processors. The various processors include, for example, a CPU which is a general-purpose processor that executes software (programs) and implements various functions. In addition, the foregoing various processors also include a graphics processing unit (GPU) which is a processor specialized in image processing and a programmable logic device (PLD) which is a processor whose circuit configuration can be modified after manufacturing, such as a field programmable gate array (FPGA). The various processors further include a dedicated electrical circuit which is a processor having a circuit configuration of a dedicated design to execute specific processing, such as an application specific integrated circuit (ASIC).

The function of each unit may be implemented by a single processor or a plurality of processors of the same type or different types (for example, a plurality of FPGAs, a combination of a CPU and a FPGA, or a combination of a CPU and a GPU). Alternatively, a single processor may implement a plurality of functions. A first example of a single processor implementing a plurality of functions is a configuration in which a combination of one or more CPUs and software composes one processor, as typified by computers such as a client and a server, and this processor implements a plurality of functions. A second example is a configuration of using a processor that implements the functions of the entire system with one integrated circuit (IC) chip, as typified by a system on a chip (SoC) and the like. As described above, various functions are implemented by using one or more processors of the various types described above as a hardware structure. Further, the hardware structures of these various processors are, more specifically, electrical circuits (circuitry) in which circuit elements such as semiconductor elements are combined.

When the foregoing processors or electrical circuits execute software (programs), code of the executed software, readable by computers (for example, various processors or electrical circuits and/or combinations of those included in the processing unit 100) is stored in a non-transitory recording medium such as ROM, and the processor refers to the software. The software that is stored in the non-transitory recording medium includes a program (identification assistance program) for executing an identification assistance method according to the present invention. The code of the program may be recorded in a non-transitory recording medium such as an optical magnetic recording device of various types or semiconductor memory, instead of in the ROM. In the case of processing using software, for example, RAM can be used as a temporary storage area, and for example, data stored in a not-illustrated electronically erasable and programmable read only memory (EEPROM) can be referred to.

<Configuration of Storing Unit>

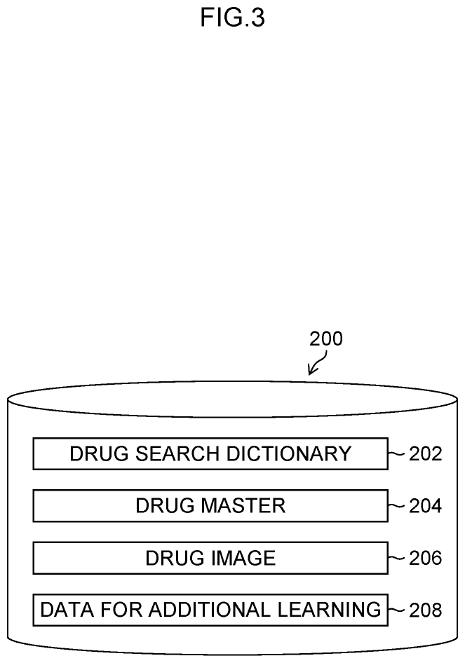

The storing unit 200 includes a non-transitory recording medium such as a Digital Versatile Disk (DVD), a hard disk, and semiconductor memory of various types and the controlling unit that controls the non-transitory recording medium. As illustrated in FIG. 3, the storing unit 200 stores a drug search dictionary 202 (drug search dictionary), a drug master 204 (drug master), drug images 206 (drug images), and data for additional learning 208. The drug search dictionary 202 is a dictionary that has learned expressions used for drug identification. For example, numerical characters (numerals), alphabet letters, and company names, trade names and abbreviated names, and other information are registered in the drug search dictionary 202 as conversion candidates. This can improve possibility that intended words are inputted as search keywords.

Identification information including codes and/or names of drugs, is associated with external appearance information on drugs, and the associated information is stored in the drug master 20, as text information. The “codes” are, for example, YJ codes (individual drug code consisting of 12 digits of alphanumeric characters), and the names may include volumes of the active ingredients. In addition, “the external appearance information” includes at least one kind of the imprint information and/or printed-letter information, the shape information, and the color information on drugs. As for imprints and printed letters, it is preferable to store information attached on both front surfaces and back surfaces of drugs. The drug master 204 may store general names of drugs and product information on the drugs, or information on original drugs (originator drugs) and information on generic drugs, with those information pieces associated with one another. The drug images 206 are stored being associated with the drug master 204. Also, as for the drug images 206, it is preferable to store information on both the front surfaces and the back surfaces of drugs.

<Configuration of Display Unit and Operation Unit>

The display unit 300 includes a monitor 310 (display device) and can display information stored in the storing unit 200, results of processing by the processing unit 100, and other information. The operation unit 400 includes: a keyboard 410 and a mouse 420 that serve as an input device and a pointing device; and a microphone 430 (voice recognizing unit) serving as a voice input device. Therefore, the user can perform operations necessary to execute the identification assistance method according to the present invention via these devices and the screen of the monitor 310 (which is described later). The monitor 310 may include a touch panel so that the user can perform operations via the touch panel.

<Processing of Identification Assistance Method>

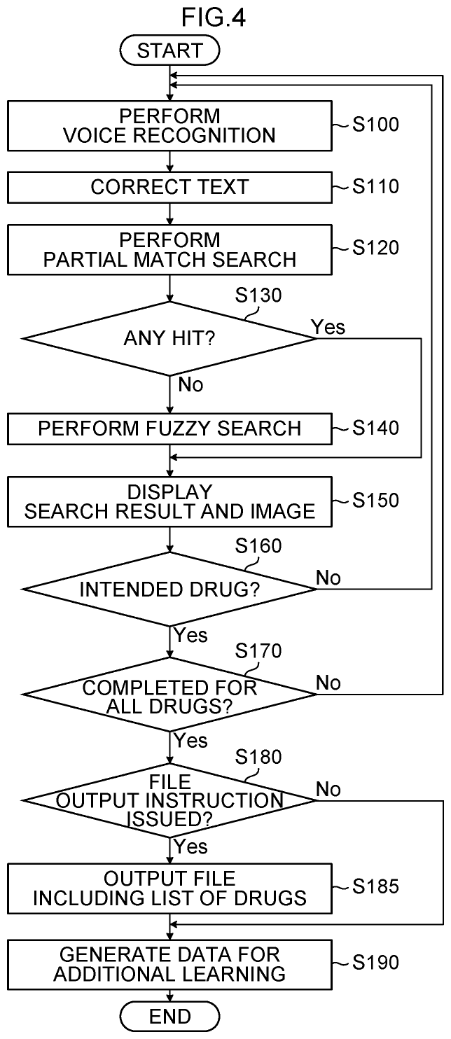

Hereinafter, an identification assistance method using the identification assistance system 10 with the foregoing configuration, is described with reference to a flowchart of FIG. 4.

<Voice Recognition>

The user reads aloud information on a drug of interest. For example, the user reads aloud, information on a generic drug, like “abcdefg tablet, 50 mg, [ABC], white” or “abcdefg, tablet, 50, [ABC], white”. The information read aloud by the user includes: a code and a name of the drug; a pharmaceutical company's name, a trade name, or abbreviated name of the pharmaceutical company; imprints and/or printed letters on the drug; a shape of the drug (a tablet or a capsule, a round shape or an oval shape, or like information); a color of the drug (an example of the external appearance information); and other information. The information read aloud may be part of the items of the foregoing information instead of all the items. In addition, the name, the imprints and/or printed letters may be read aloud partially. In addition, the name of the drug, the pharmaceutical company's name, the trade name, or the abbreviated name of the pharmaceutical company, and the imprints and/or printed letters may be the ones attached on a package (PTP sheet or the like) (PTP: Press Through Pack) of the drug. The microphone 430 receives input of the voice, and the voice recognizing unit 102 recognizes the received voice and outputs the recognition result as first text (step S100: voice recognition process). The voice recognizing unit 102 is configured to recognize and output one or more words. In a case where the voice recognizing unit 102 recognizes a word, after a lapse of a certain time with no received voice, the voice recognizing unit 102 can take the word as another new word.

<Correction of Text>

Because a general voice recognition model is based on assumption of generally used words, there is a possibility that drug identification outputs words (text) different from intended words. To address this, the text correcting unit 104 according to the first embodiment refers to the drug search dictionary 202 (drug search dictionary) that has learned expressions used for drug identification, and corrects the first text to generate second text (step S110: text correction process). The drug search dictionary 202 (drug search dictionary) is a conversion dictionary in which words used for drug identification are registered as conversion candidates. For example, numerical characters (numerals), alphabet letters, the pharmaceutical companies' names, trade names, or abbreviated names of pharmaceutical companies, and other information are registered in the drug search dictionary 202. In some cases, such information is attached to drugs by using imprints and/or printed letters, print on packages of the drugs, attachment of labels to the packages, and by other methods. Thus, because such information is registered into the drug search dictionary 202, it is possible to receive user's intended words as search keywords and perform accurate search. Note that the text correcting unit 104 may be configured so as to receive correction to the second text and cause the drug search dictionary 202 to perform additional learning based on the received correction (which is described later).

<Search>

The searching unit 106, as described in detail below, performs a partial match search using the second text as keywords (step S120: search process, partial match search process). In addition, depending on the results of the partial match search, the searching unit 106 performs a fuzzy search (steps S130, 140: search process, fuzzy search process).

<Normalization of Text>

The searching unit 106 normalizes the second text to generate normalized text, and using the normalized text, performs a partial match search (step S120: search process, normalization process, partial match search process). As “normalization”, for example, the searching unit 106 can perform conversion (or conversion in the reverse direction) from uppercase letters to lowercase letters, from full-width characters to half-width characters, and from kanji characters (Chinese characters) and/or hiragana characters (rounded Japanese phonetic syllabary) to katakana characters (angular Japanese phonetic syllabary). Therefore, it becomes possible to unify expression of the texts to improve search accuracy. Preferably, the searching unit 106 performs conversion according to the expression formats of the identification information in the drug master 204 (for example, whether uppercase letters are used or lowercase letters are used).

<Partial Match Search>

At step S120, the searching unit 106 performs a partial match search by searching the drug master 204 using the second text about the drug name, the imprints and/or printed letters (on each of the front surface and the back surface), and the like as keywords (if there are multiple keywords, the searching unit 106 performs an AND search of multiple keywords) and calculates the agreement degree. The searching unit 106 sorts the search results by the agreement degree, and regards the drugs having agreement degrees larger than or equal to a threshold, as candidate drugs (candidates of drugs indicated by the second text). Then, the searching unit 106 obtains the identification information including the codes and/or names of the candidate drugs (which may include information on the imprints and/or printed letters) and the images corresponding to the identification information from the storing unit 200 (drug database) (step S120). The searching unit 106 may calculate, as “the agreement degree”, “the matching rate (=the number of matched characters/the total number of all the characters)” and/or “the agreement position rate (=the position of the first character in agreement/the total number of all the characters)”.

<Fuzzy Search>

The searching unit 106 performs a fuzzy search depending on the results of the partial match search. For example, the searching unit 106 determines whether any hit has been returned by the partial match search (whether one or more candidate drugs have been returned) (step S130: search process). If there is no hit (NO at step S130), the searching unit 106 performs a fuzzy search (step S140).

At step S140, the searching unit 106 calculates similarity degree between the text (the second text) corrected at step S110 and the text information (identification information, external appearance information; third text) stored in the drug master 204, and obtains the identification information and image of the drugs (candidate drugs) whose similarity degrees are larger than or equal to a threshold (search process, fuzzy search process). The searching unit 106 can use the Levenshtein distance, the Damerau-Levenshtein distance, the Hamming distance, the Jaro-Winkler distance, and the like as an indicator indicating the similarity degree of text (character strings).

<Calculation of Similarity Degree in Consideration of Number of Characters of Keyword>

In a case where identification is performed through voice input of the drug's name or the like, the user often reads aloud only part of the name or the like instead of the whole name, and as a result, the keyword is often shortened. In this case, the similarity degree of a shorter drug's name to the keyword is relatively higher than that of a longer drug's name, and this makes it impossible to obtain appropriate search results in some cases. To address this, the identification assistance system 10 can calculate the similarity degree in consideration of the number of characters of the keyword, as described in the following. Specifically, in a case where “the number of characters of corrected text (second text)” is smaller than “the number of characters in the text information (third text) stored in the drug master 204”, the searching unit 106 extracts one or more character strings having the same length as the second text from the third text, calculates the similarity degree between the one or more extracted character strings and the second text, and uses the value for the case in which the similarity degree is largest (step S140: search process, fuzzy search process). On the other hand, in a case where “the number of characters of corrected text” is larger than or equal to “the number of characters in the text information stored in the drug master 204”, the searching unit 106 does not perform the extraction of one or more character strings, and calculates the similarity degree between the third text as is and the second text (step S140: search process, fuzzy search process).

Thus, since the number of characters of the keyword is taken into consideration when calculating the similarity degree, it becomes easier to obtain accurate search results can be obtained.

<Search Result and Display of Image>

The outputting unit 108 displays (outputs) the identification information and image of the candidate drug(s) on the monitor 310 (display device) (step S150: output process). With the display of the identification information and the image, the user can understand easily whether the search result is the drug that the user intends. In a case in which the identification assistance system 10 (searching unit 106) determines that “the candidate drug is not the drug that the user intends” (NO at step S160) and in a case in which the identification assistance system 10 determines that “searching for all the drugs has not been completed” (NO at step S170), the identification assistance system 10 returns to step S100 and repeats the processing. The identification assistance system 10 can make these determinations based on the operation by the user via the operation unit 400.

<File Output of Search Result>

In a case in which the identification assistance system 10 (searching unit 106) determines that “the candidate drug is the drug that the user intends” (YES at step S160) and also determines that “searching for all the drugs has been completed” (YES at step S170), the outputting unit 108 determines whether a file output instruction for output the search results has been issued (step S180: file output process). In a case in which the file output instruction has been issued, the outputting unit 108 outputs a file including the identification information on the drug (information including the code and/or the name of the drug) selected from the candidate drugs (step S185: file output process). The outputting unit 108 may store the file in the storing unit 200. The outputting unit 108 can determine whether the file output instruction has been issued and which drug has been selected, based on the operation by the user via the operation unit 400. Note that the outputted file can be utilized in other systems such as a brought-in-drug order system.

<Additional Learning>

The text correcting unit 104 can receive correction to the second text according to an instruction by the user via the operation unit 400 and cause the drug search dictionary 202 to perform additional learning based on the received correction. Examples of possible additional learning include: updating the drug search dictionary 202 using the corrected text (words); and making a trained model (which is described later) perform additional learning using corrected text as teacher data. When the text correcting unit 104 receives correction to the second text, the text correcting unit 104 generates data for additional learning 208 according to the details of received correction (step S190: data generation process). The text correcting unit 104 may be configured to cause the drug search dictionary 202 to perform the additional learning every time when the text correcting unit 104 generates data for additional learning; or to cause the drug search dictionary 202 to perform the additional learning periodically or at any time according to an instruction by the user via the operation unit 400. The additional learning improves accuracy in generating the first and second text.

<Advantageous Effect of First Embodiment>

As has been described above, with the identification assistance system 10 and the identification assistance method according to the first embodiment, the user can identify drugs accurately and easily.

<Generation of Text by Trained Model>

In the first embodiment, description has been made based on a configuration in which the text correcting unit 104 refers to the drug search dictionary 202 to correct the voice recognition result (first text). However, the identification assistance system according to the present invention may be configured so as to generate the first text by using a trained model built by machine learning using the identification information and the external appearance information as teacher data. Such a trained model can be built by using a recurrent neural network (RNN: one mode of a neural network) based on a natural-language processing algorithm. The RNN is different from other neural networks (such as a convolution neural network) in that the RNN has an input layer, a hidden layer, and an output layer, and that the hidden layer has a first hidden layer indicating the state of the current time (time t) and a second hidden layer indicating the state of the past time (time t−1). A trained model of the RNN holds the state of the hidden layer at time t−1 and uses it for the input at the next time t, so that it is possible to perform inference (estimation) using past histories of information (order of characters or words in the voice recognition in the present embodiment) that is inputted chronologically like a natural language. Here, the trained model may be built by using long short-term memory (LSTM), which is a type of RNN.

### Second Embodiment

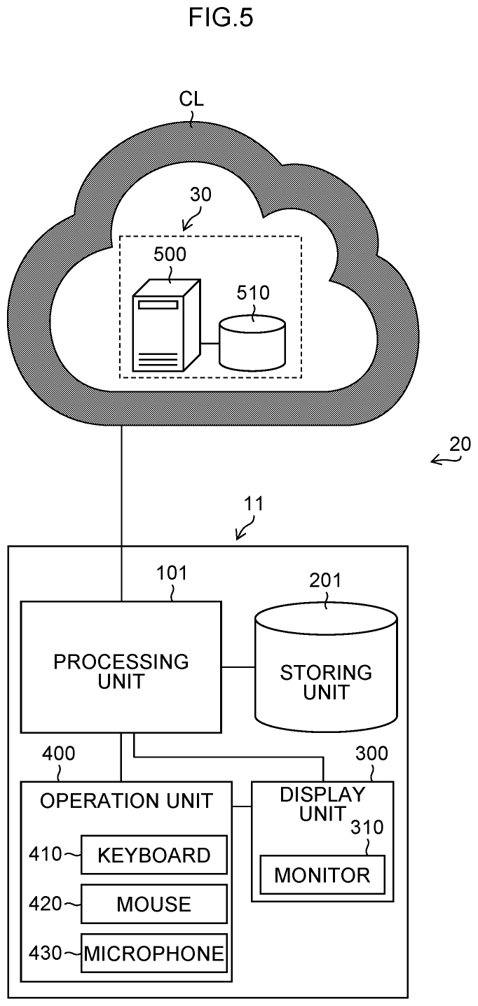

FIG. 5 is a diagram illustrating a configuration of an identification assistance system 20 (identification assistance system) according to a second embodiment of the present invention. The identification assistance system 20 has functions the same as or similar to those of the identification assistance system 10 according to the first embodiment as a whole, but is different from that of the first embodiment in that the system includes an identification assistance client 11 (identification assistance client) and an identification assistance server 30 (identification assistance server). Note that, as for the identification assistance system 20, the constituents common to those of the identification assistance system 10 according to the first embodiment are denoted by the same reference numerals, and detailed description thereof is omitted.

<Configuration of Identification Assistance Client>

The identification assistance client 11 includes: a processing unit 101; a storing unit 201; a display unit 300; and an operation unit 400. The identification assistance client 11 performs, as described later, voice recognition, data transmission and reception to and from the identification assistance server 30, processing result display, and other operations. The identification assistance client 11 can be realized by using a computer such as a personal computer or a portable terminal such as a smartphone. The display unit 300 and the operation unit 400 may be integrated by using a monitor of a touch-panel type.

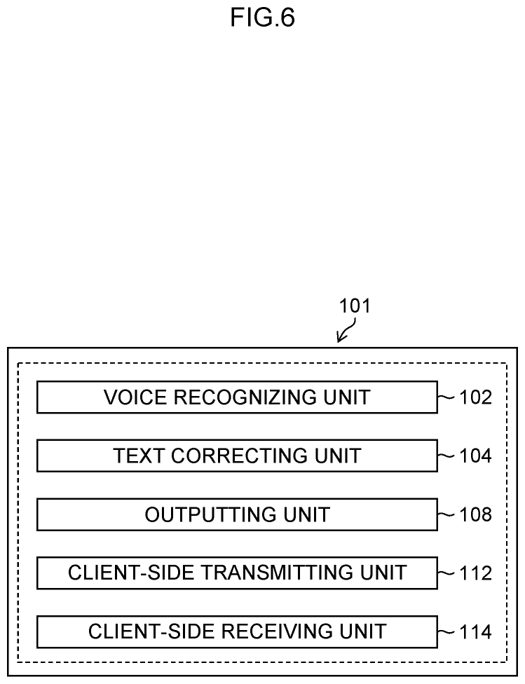

FIG. 6 is a diagram illustrating a functional configuration of the processing unit 101. The processing unit 101 includes: a voice recognizing unit 102 (voice recognizing unit); a text correcting unit 104 (text correcting unit); an outputting unit 108 (outputting unit); a client-side transmitting unit 112 (client-side transmitting unit); and a client-side receiving unit 114 (client-side receiving unit). These units can be implemented with various processors and electrical circuits as described about the processing unit 100 above. In a case where a processor or an electrical circuit executes software (programs), ROM, RAM, and the like may be used.

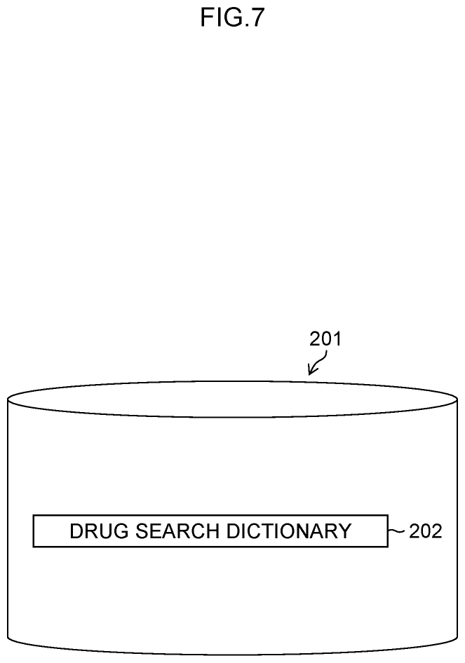

FIG. 7 is a diagram illustrating a configuration of the storing unit 201. The storing unit 201 stores therein, a drug search dictionary 202 (see FIG. 3) and data for additional learning 208 (see FIG. 3).

<Configuration of Identification Assistance Server>

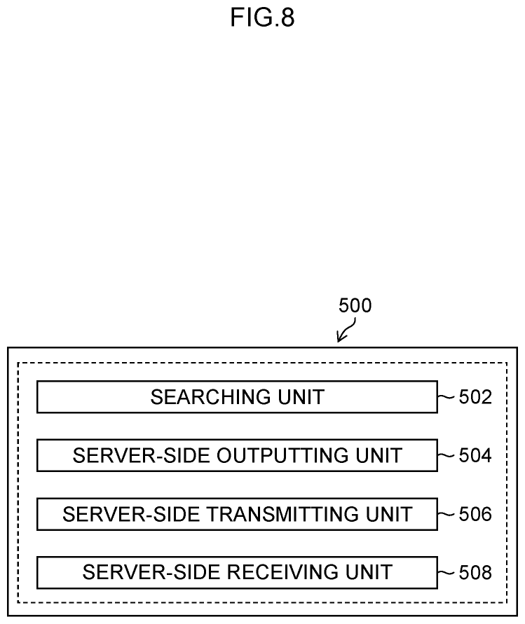

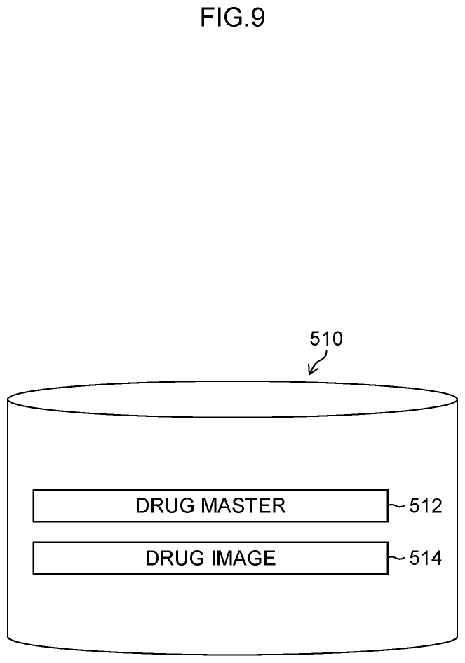

The identification assistance server 30 is a server on a cloud CL (see FIG. 5) and includes a server main unit 500 and a storing unit 510 (drug database). The server main unit 500, as illustrated in FIG. 8, includes: a searching unit 502 (searching unit); a server-side outputting unit 504 (server-side outputting unit); a server-side transmitting unit 506 (server-side transmitting unit); and a server-side receiving unit 508 (server-side receiving unit). As illustrated in FIG. 9, the storing unit 510 stores therein: a drug master 512 (which is the same as or similar to the drug master 204 in FIG. 3); and drug images (which are the same as or similar to the drug images 206 in FIG. 3).

<Processing by Identification Assistance Method>

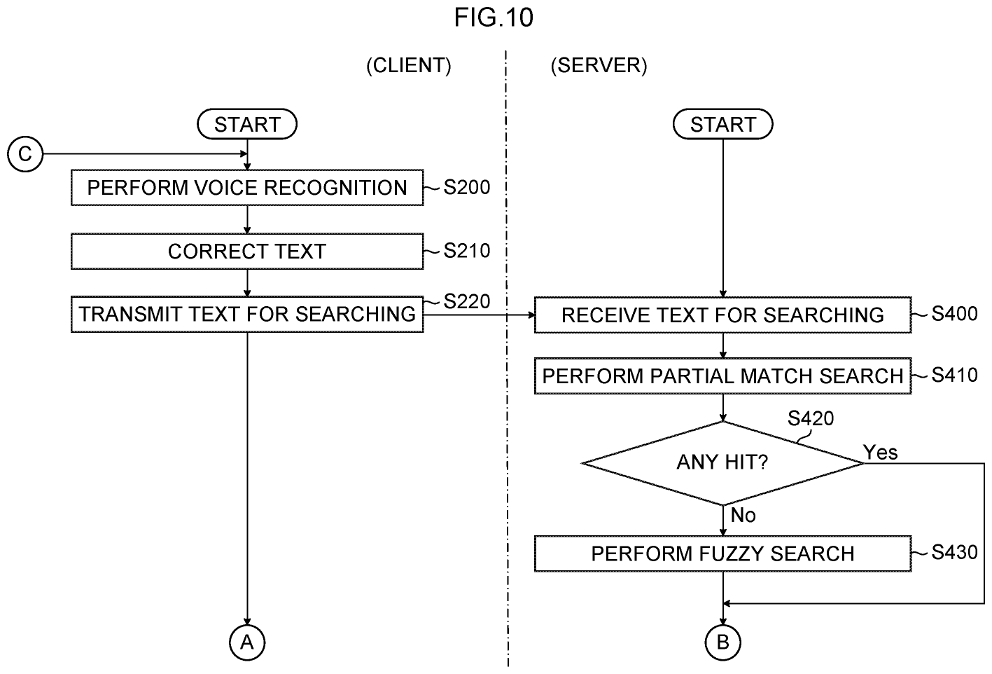

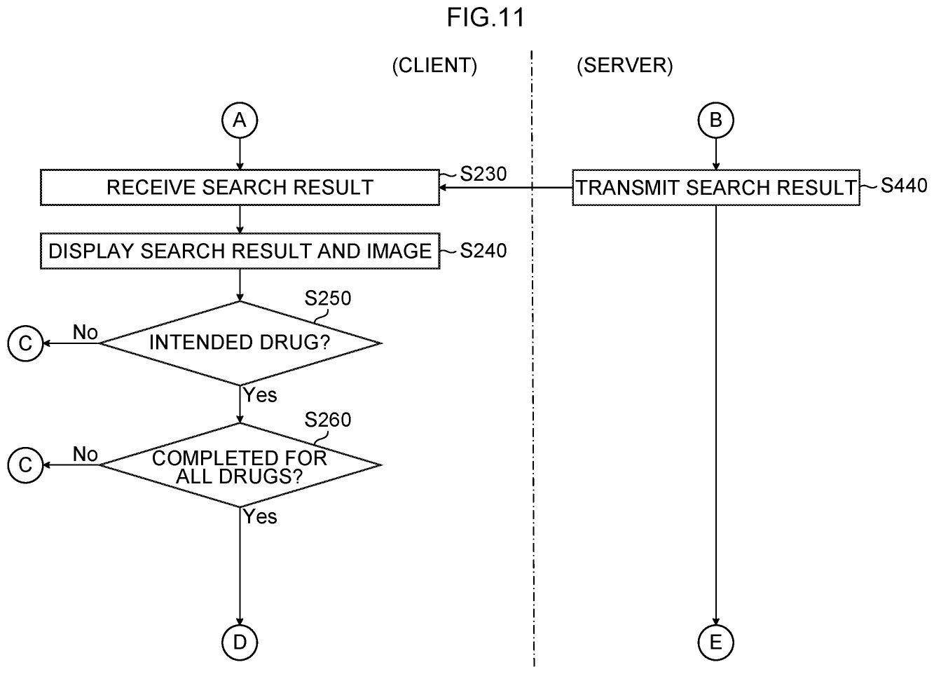

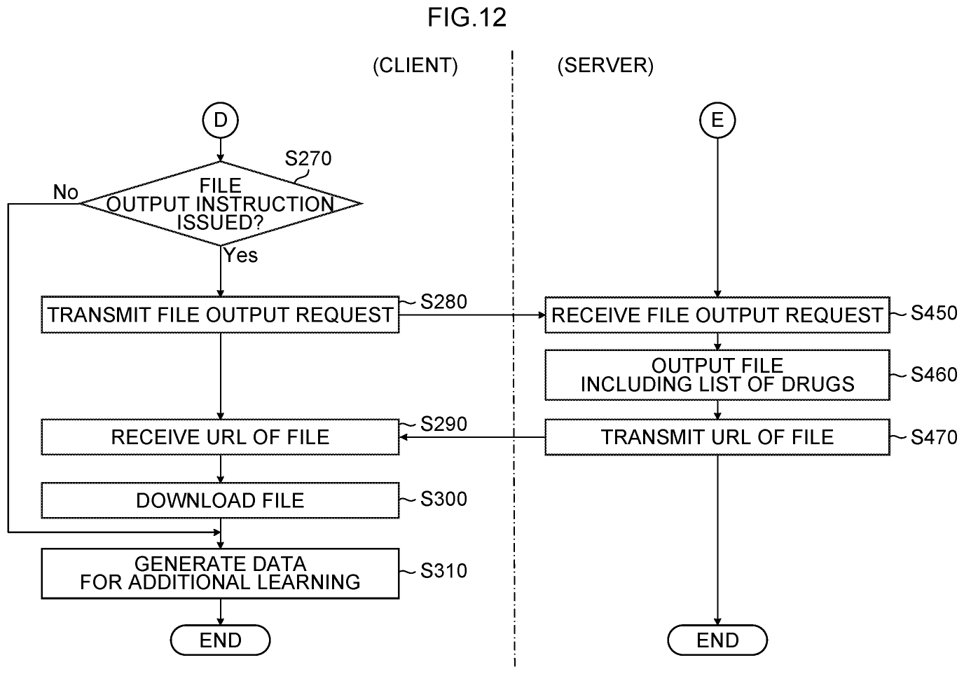

FIGS. 10 to 12 are flowcharts illustrating processing by the identification assistance method according to the second embodiment. In these figures, the left sides illustrate processing in the identification assistance client 11, and the right sides illustrate processing in the identification assistance server 30. The voice recognizing unit 102 and the text correcting unit 104 of the identification assistance client 11 execute the processing in steps S200 and S210, as in the steps S100 and S110 in the first embodiment (generation of first text by voice recognition and generation of second text by correcting the text; voice recognition process and text correction process). The text correcting unit 104, as in the first embodiment, may generate text by using a trained model. The client-side transmitting unit 112 transmits text information on the drug (text for searching; second text) to the identification assistance server 30 (step S220), and the server-side receiving unit 508 (server-side receiving unit) of the identification assistance server 30 receives the text information (step S400).

The searching unit 502, as in the steps S120 to S140, searches the drug master 512 (drug database) using the received text information as keywords and obtains the identification information and images of candidate drugs (steps S410 to S430; search process, normalization process, partial match search process, and fuzzy search process). The server-side transmitting unit 506 transmits the search results (identification information and images) to the identification assistance client 11 (step S440). Then, the client-side receiving unit 114 receives the search results (step S230), and the outputting unit 108 displays the identification information and the images of the candidate drugs on the monitor 310 (display device) (step S240: output process). The identification assistance client 11, as in the steps S160 to S190, repeats the processing in steps S200 to S250 until the processing finishes for all the drugs (until the determination at step S260 becomes YES).

Note that description of the second embodiment is made based on a case in which the storing unit 510 of the identification assistance server 30 stores drug images (drug images 514) in consideration of system load on the identification assistance client 11. However, if the processing capability of the identification assistance client 11 is high enough, the storing unit 201 of the identification assistance client 11 may store drug images therein.

The outputting unit 108 determines whether a file output instruction for outputting the search results has been issued (step S270: file output process). In a case where the outputting unit determines that the file output instruction has been issued, the client-side transmitting unit 112 transmits a file output request to the identification assistance server 30 (step S280: file output process), and the server-side receiving unit 508 receives the file output request (step S450). The server-side outputting unit 504, in response to the reception of the file output request, outputs a file including identification information on the drug selected out of the candidate drugs (information including the code and/or the name of the drug) (step S460: file output process), and the server-side transmitting unit 506 transmits a uniform resource locator (URL) indicating a place where the file is stored to the identification assistance client 11 (step S470). The place where the file is stored may be the storing unit 510 or may be another storing device. The client-side receiving unit 114 receives the URL (step S290), and the outputting unit 108 downloads the file from the specified URL (step S300). The outputting unit 108 may store the downloaded file into the storing unit 200.

The text correcting unit 104 of the identification assistance client 11, as in step S190, generates data for additional learning (step S310).

As has been described above, also with the identification assistance system and identification assistance method according to the second embodiment, the user can identify drugs accurately and easily, as in the first embodiment.

Although the embodiments of the present invention and other examples have been described above, the present invention is not limited to the foregoing aspects, but various modification can be made within the scope not departing from the spirit of the present invention.

### EXPLANATION OF REFERENCES

10 identification assistance system
11 identification assistance client
20 identification assistance system
30 identification assistance server
100 processing unit
101 processing unit
102 voice recognizing unit
104 text correcting unit
106 searching unit
108 outputting unit
110 communication controlling unit
112 client-side transmitting unit
114 client-side receiving unit
200 storing unit
201 storing unit
202 drug search dictionary
204 drug master
206 drug image
208 data for additional learning
300 display unit
310 monitor
400 operation unit
410 keyboard
420 mouse
430 microphone
500 server main unit
502 searching unit
504 server-side outputting unit
506 server-side transmitting unit
508 server-side receiving unit
510 storing unit
512 drug master
514 drug image
CL cloud
S100 to S470 steps of identification assistance method

What is claimed is:

1. An identification assistance system comprising:
a voice recognizing unit configured to recognize received voice and output a recognition result as first text;
a text correcting unit configured to refer to a drug search dictionary having learned expressions used for drug identification, and correct the first text to generate second text;
a drug database configured to store identification information including codes and/or names of drugs and external appearance information on the drugs, as text information in a state where the external appearance information is associated with the identification information;
a searching unit configured to search the drug database using the second text as a keyword and obtain the identification information on at least one candidate drug that is a candidate of a drug indicated by the second text; and
an outputting unit configured to output the identification information on the candidate drug, wherein
the searching unit performs a partial match search using the second text as the keyword and performs a fuzzy search depending on a result of the partial match search,
in the fuzzy search, the searching unit calculates a similarity degree between the second text and third text that is text included in the text information, and regards a drug that corresponds to the third text whose similarity degree is larger than or equal to a threshold, as the candidate drug, and
the searching unit extracts a character string having the same length as the second text out of the third text and calculates the similarity degree.

2. The identification assistance system according to claim 1, wherein the drug search dictionary is a conversion dictionary in which words used for drug identification are registered as conversion candidates.

3. The identification assistance system according to claim 1, wherein the voice recognizing unit generates the first text, using a trained model built by machine learning performed using the identification information and the external appearance information as teacher data.

4. The identification assistance system according to claim 1, wherein the searching unit normalizes the second text to generate normalized text and uses the normalized text to perform the partial match search.

5. The identification assistance system according to claim 1, wherein the text correcting unit receives correction to the second text and causes additional learning of the drug search dictionary based on the received correction.

6. The identification assistance system according to claim 1, wherein the external appearance information includes at least one kind of information out of imprint information and/or printed-letter information, shape information, and color information on the drugs.

7. The identification assistance system according to claim 1, wherein the outputting unit outputs a file including the identification information on a drug selected out of the candidate drug.

8. The identification assistance system according to claim 1, wherein
the drug database stores the identification information on the drugs and images of the drugs, in a state where the images are associated with the identification information, and
the outputting unit outputs an image of the candidate drug with the image of the candidate drug associated with the identification information, to a display device.

9. An identification assistance system comprising an identification assistance server, and an identification assistance client connected to the identification assistance server via a network, wherein
the identification assistance client comprises:
a voice recognizing unit configured to recognize received voice and output a recognition result as first text;
a text correcting unit configured to refer to a drug search dictionary having learned expressions used for drug identification, and correct the first text to generate second text;
a client-side transmitting unit configured to transmit information indicating the second text to the identification assistance server;
a client-side receiving unit configured to receive identification information on at least one candidate drug that is a candidate of a drug corresponding to the second text from the identification assistance server, the identification information including a code and/or a name of the drug; and
an outputting unit configured to output the identification information,
the identification assistance server comprises:
a drug database configured to store identification information including codes and/or names of drugs and external appearance information on the drugs, as text information in a state where the external appearance information is associated with the identification information;
a server-side receiving unit configured to receive the information indicating the second text from the identification assistance client;
a searching unit configured to search the drug database using the second text as a keyword and obtain the identification information on at least one candidate drug that is a candidate of a drug indicated by the second text; and
a server-side transmitting unit configured to transmit the obtained identification information to the identification assistance client,
the searching unit performs a partial match search using the second text as the keyword and performs a fuzzy search depending on a result of the partial match search,
in the fuzzy search, the searching unit calculates a similarity degree between the second text and third text that is text included in the text information stored in the drug database, and regards a drug that corresponds to the third text whose similarity degree is larger than or equal to a threshold, as the candidate drug, and
the searching unit extracts a character string having the same length as the second text out of the third text and calculates the similarity degree.

10. The identification assistance system according to claim 9, wherein
in the identification assistance client, the drug search dictionary is a conversion dictionary in which words used for drug identification are registered as conversion candidates.

11. The identification assistance system according to claim 9, wherein
in the identification assistance client, the voice recognizing unit generates the first text, using a trained model built by machine learning performed using the identification information and the external appearance information as teacher data.

12. The identification assistance system according to claim 9, wherein
in the identification assistance server, the searching unit normalizes the second text to generate normalized text and uses the normalized text to perform the partial match search.

13. The identification assistance system according to claim 9, wherein
in the identification assistance client, the text correcting unit receives correction to the second text and causes additional learning of the drug search dictionary based on the received correction.

14. The identification assistance system according to claim 9, wherein
in the drug database in the identification assistance server, the external appearance information includes at least one kind of information out of imprint information and/or printed-letter information, shape information, and color information on the drugs.

15. The identification assistance system according to claim 9, wherein
in the identification assistance client, the outputting unit outputs a file including the identification information on a drug selected out of the candidate drug.

16. The identification assistance system according to claim 9, wherein
in the identification assistance server, the drug database stores the identification information on the drugs and images of the drugs, in a state where the images are associated with the identification information, and
in the identification assistance client, the outputting unit outputs an image of the candidate drug with the image of the candidate drug associated with the identification information, to a display device.

17. An identification assistance server comprising:
a drug database configured to store identification information including codes and/or names of drugs and external appearance information on the drugs, as text information in a state where the external appearance information is associated with the identification information;
a server-side receiving unit configured to receive information indicating a second text on a drug from an identification assistance client;
a searching unit configured to search the drug database using the second text as a keyword and obtain the identification information on at least one candidate drug that is a candidate of the drug indicated by the second text; and
a server-side transmitting unit configured to transmit the obtained identification information to the identification assistance client, wherein
the searching unit performs a partial match search using the second text as the keyword and performs a fuzzy search depending on a result of the partial match search,
in the fuzzy search, the searching unit calculates a similarity degree between the second text and third text that is text included in the text information stored in the drug database, and regards a drug that corresponds to the third text whose similarity degree is larger than or equal to a threshold, as the candidate drug, and
the searching unit extracts a character string having the same length as the second text out of the third text and calculates the similarity degree.

18. An identification assistance method to be performed by at least one computer, comprising:
recognizing received voice and outputting a recognition result as first text;
referring to a drug search dictionary having learned expressions used for drug identification and correcting the first text to generate second text;
searching, using the second text as a keyword, a drug database storing identification information including codes and/or names of drugs and external appearance information on the drugs, as text information, in a state where the external appearance information is associated with the identification information, and obtaining the identification information on at least one candidate drug that is a candidate of a drug indicated by the second text; and
outputting the identification information on the candidate drug, wherein
in the searching, a partial match search is performed using the second text as the keyword and a fuzzy search is performed depending on a result of the partial match search,
in the fuzzy search in the searching, a similarity degree is calculated between the second text and third text that is text included in the text information stored in the drug database, and a drug that corresponds to the third text whose similarity degree is larger than or equal to a threshold, is regarded as the candidate drug, and
in the searching, a character string having the same length as the second text is extracted out of the third text to calculate the similarity degree.

19. An identification assistance method to be performed by at least one computer, comprising:
receiving information indicating a second text on a drug from an identification assistance client;
searching, using the second text as a keyword, a drug database which stores identification information including codes and/or names of drugs and external appearance information on the drugs, as text information in a state where the external appearance information is associated with the identification information;
obtaining the identification information on at least one candidate drug that is a candidate of the drug indicated by the second text; and
transmitting the obtained identification information to the identification assistance client, wherein
in the searching, a partial match search is performed using the second text as the keyword and a fuzzy search is performed depending on a result of the partial match search,
in the fuzzy search in the searching, a similarity degree is calculated between the second text and third text that is text included in the text information stored in the drug database, and a drug that corresponds to the third text whose similarity degree is larger than or equal to a threshold, is regarded as the candidate drug, and
in the searching, a character string having the same length as the second text is extracted out of the third text to calculate the similarity degree.
